(19) United States
(12) Patent Application Publication
(10) Pub. No.: US 2024/0252222 A1
(43) Pub. Date: Aug. 1, 2024

(54) METHODS AND DEVICES FOR ELECTROPORATION FOR TREATMENT OF VENTRICULAR FIBRILLATION

(71) Applicant: Mayo Foundation for Medical Education and Research, Rochester, MN (US)

(72) Inventors: Christopher V. DeSimone, Rochester, MN (US); Samuel J. Asirvatham, Rochester, MN (US); Christopher J. McLeod, Ponte Vedra Beach, FL (US); Jason A. Tri, Rochester, MN (US)

(21) Appl. No.: 18/565,650

(22) PCT Filed: May 26, 2022
(86) PCT No.: PCT/US2022/031026

§ 371 Date: Nov. 30, 2023

Related U.S. Application Data
(60) Provisional application No. 63/195,420, filed on Jun. 1, 2021.

(51) Int. Cl. A61B 18/00

(52) U.S. Cl. CPC A61B 18/00; A61B 2018/00065; A61B 2018/00238; A61B 2018/00357; A61B 2018/00363; A61B 2018/00386; A61B 2018/00404; A61B 2018/00434; A61B 2018/00541; A61B 2018/00577; A61B 2018/00613; A61B 2018/00648; A61B 2018/00708; A61B 2018/00761; A61B 2018/00839; A61B 2018/00875; A61B 2018/00994

(57) ABSTRACT

This document describes methods and materials for treating ventricular fibrillation. For example, this document describes methods and devices for delivering pulse-electric field electroporation for ablation in a selective and non-thermal means that has high specificity for tissue destruction while minimizing collateral damage. In one aspect, this document is directed to a catheter device that includes: a catheter shaft; a balloon member attached at a distal end portion of the catheter shaft; one or more electrodes on an outer surface of the balloon or in an interior of the balloon member and configured to deliver pulsed-electric field non-thermal electroporation ablation energy to treat ventricular fibrillation of a heart; and a control system configured to adjust a pressure within the balloon member.

### CROSS-REFERENCE TO RELATED APPLICATIONS

This application claims the benefit of U.S. Provisional Application Ser. No. 63/195,420, filed Jun. 1, 2021. The disclosure of the prior application is considered part of (and is incorporated by reference in) the disclosure of this application.

### BACKGROUND

### 1. Technical Field

This document relates to methods and materials for treating ventricular fibrillation. For example, this document relates to methods and devices for delivering pulsed-electric field electroporation for ablation in both a selective and as a non-thermal means, with high tissue specificity for destruction while minimizing collateral damage to critical structures of the heart and extracardiac structures.

### 2. Background Information

Ventricular fibrillation (also referred to herein as “VF”) is a lethal rhythm that can result in sudden cardiac death (SCD). This is the number one cause of death—greater than all deaths from cancer in the United States combined. There is no cure for ventricular fibrillation that can lead to SCD—only treatments which are aimed at prevention of SCD such as drug therapy (which may be ineffective and fraught with side effects). ICD (“implantable cardiac defibrillator”) therapy is protective and could shock the patient back into normal rhythm, but also portends patients to ineffective shocks, inappropriate shocks, as well as post-traumatic stress disorder from receiving shock therapy. Radiofrequency (RF) ablation is limited in efficacy and issues with thermal ablation could lead to complications and unwanted tissue destruction. Although defibrillators, anti-arrhythmics, and other therapies provide an element of protection in select cases, sudden cardiac death remains a major worldwide health problem.

Electroporation is a technique that uses very brief pulses of high voltage to introduce multiple nanopores within the cells' wall in a non-thermal manner (unlike RF), specifically within the lipid bilayer of the cell membranes as a result of the change in electrical field. Depending on the voltage and frequency of pulsations used, these pores can be reversible (i.e., increase the permeability of these cell to chemotherapeutic agents) and or irreversible (i.e., trigger cell death by the process of apoptosis or necrosis). Given the different composition of each cell-type membrane, electroporation can allow for a differential effect on different tissues.

### SUMMARY

This document describes methods and materials for treating ventricular fibrillation. For example, this document describes methods and devices for delivering pulsed-electric field electroporation for ablation in a selective and non-thermal means with high specificity for tissue destruction while minimizing collateral damage.

In one aspect, this disclosure is directed to a catheter device that includes: a catheter shaft; a balloon member attached at a distal end portion of the catheter shaft; one or more electrodes on an outer surface of the balloon or in an interior of the balloon member and configured to deliver pulsed-electric field non-thermal electroporation ablation energy to treat ventricular fibrillation of a heart; and a control system configured to adjust a pressure within the balloon member.

Such a catheter device may optionally include one or more of the following features. The control system may be configured to pressurize and depressurize the balloon member in synchronization with an ECG of the heart. The control system may be configured to pressurize and depressurize the balloon member based on a rate of change of pressure in a left ventricular cavity of the heart such as that from cardiac contraction. The control system may be configured to pressurize and depressurize the balloon member based on a timing cycle. The control system may be configured to pressurize and depressurize the balloon member based on impedance changes in the heart. The control system may be configured to pressurize and depressurize the balloon member based on impedance changes of a target tissue so as to automatically shut-off when ablation is completed. The control system may be configured to pressurize and depressurize the balloon member based on a timing in order to inflate for a period and deflate for a specified time in order to allow for adequate perfusion of the heart and maintenance of blood pressure. The control system may be configured to pressurize and depressurize the balloon member in coordination with the heart's systole/diastole in order to augment ventricular output. The balloon member may comprise an inner balloon with pores that is configured to inflate to a certain desired amount. The balloon member may comprise a porous material in order to not constrict or impede blood flow in the heart that would otherwise lead to hemodynamic instability.

In another aspect, this disclosure is directed to a method for treating ventricular fibrillation of a heart. The method includes using one or more of the catheter devices described above to deliver pulsed-electric field non-thermal electroporation ablation energy to one or more target locations of the heart and using these unique vantage points to render VF terminated or non-inducible. a

Such a method may optionally include one or more of the following features. The one or more target locations of the heart may include a right stellate ganglion, a left stellate ganglion, or both. The one or more target locations of the heart may include an endocardial space of the heart and an epicardial space of the heart. The one or more target locations of the heart may include an arch of an aorta of the heart and associated ganglia The one or more target locations of the heart may include around great arteries of the heart and associated ganglia. The one or more target locations of the heart may include in a pericardial sac of the heart and in an oblique or transverse sinus of the heart and its associated ganglia. The one or more target locations of the heart may include the superior mediastinum and its associated nerves and ganglia which encases the heart. The one or more target locations of the heart may include an azygous vein of the heart. The one or more target locations of the heart may include an aorta of the heart. The method may also include placing one or more electrodes in a left or right mainstem bronchus to stimulate or ablate cardiac or nerve tissue related to the heart and heart rhythm. The one or more target locations of the heart may include in a superior intercostal vein. The method may also include placing one or more electrodes in a trachea or endotracheal tube to stimulate or ablate cardiac or nerve tissue related to the heart and heart rhythm

In another aspect, this disclosure is directed to a method of pace termination for treating ventricular fibrillation (VF) of a heart. The method includes delivering pacing to one or more target locations of the heart during one or more specific timings of the VF. The pacing delivered may be based on the target locations and/or a time of the VF. The pacing delivered may be based on duration of VF with the use of hemodynamic support in order to keep critical tissues alive.

Such a method of pace termination may optionally include one or more of the following features. The one or more target locations of the heart may include the endocardium of the heart; and the epicardium of the heart. The one or more target locations of the heart may include the interventricular septum of the heart. The pacing may be delivered using a screw-in coil positioned longitudinally into the interventricular septum. The one or more target locations of the heart may include one or more of the following: (i) a distal coronary artery of the heart, (ii) a distal coronary venous branch, (ii) a mid-myocardium of the heart, and (iv) a papillary muscle head of the heart.

Particular embodiments of the subject matter described in this document can be implemented to realize one or more of the following advantages. The methods and devices for electroporation can limit damage to cardiac conduction tissue, coronary arteries and veins, phrenic nerve, cardiac valves, cardiac ganglia, and normal cardiac muscle by selectively targeting tissues for ablation based on differences in tissue response. The systems and methods can provide superficial ablations that are far reaching to accommodate variations in the shape of the ventricle and wide-areas of desired tissue effects. Similarly, the systems and methods can provide these ablation lesions to occur in proximal and distal regions of the specialized conduction tissue of the heart (His-purkinje system), as well as distal or proximal only. In some cases, the systems and methods themselves can provide hemodynamic support similar to an Impella, cardiopulmonary bypass, or Extracorporeal membrane oxygenation (ECMO) circuit by using the catheter and shaft as ports for inflow and outflow of blood and/or other fluids. Such a method can enhance catheter stability as well as hemodynamic support during mapping and ablation of tissue. Alternatively, deeper and more specific ablation can also be provided. Superficial and deeper lesions could lead to treatments that can include, but are not limited to, ventricular fibrillation ablation, ventricular tachycardia ablation, pre-mature ventricular contraction ablation, myocardial specific ablation, and/or ganglia ablation. Treatment can also include vessel avoidance and provide modulation of treatment. Further, ablation can be provided with minimal or no damage to healthy heart tissue. As the Purkinje network is involved in both the triggering and sustainment of ventricular arrhythmias including VF, modifying the Purkinje cell membrane potential via a drug (e.g., through the mechanism of action of the drug, or a delivery of the drug) can provide a means to terminate arrhythmias through slowing electrical conduction or prolonging cell refractoriness, especially specific to the conduction system, as a means to modulate, terminate, or enhance ablation efficacy or sparing of conduction tissue. Catheter device embodiments described herein can include the ability to access the right and left ventricle safely, while negotiating the valve apparatus safely. Furthermore, large regions of ventricular and His-Purkinje (HPS) specialized conduction tissue can be obtained quickly and with good contact. Finally, some designs have the all-in-one ability for the device electrodes to provide the ability for conformational chamber design and can perform mapping, pacing, and electroporation ablation of ventricular myocardium and/or the HPS in order to address all the needs for success.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings and from the claims.

### DESCRIPTION OF DRAWINGS

FIG. 1 is a cross-sectional view of a human heart.

FIG. 2 illustrates an example of regional variability of intracardiac electrograms during ventricular fibrillation.

FIGS. 3A and 3B are examples of ventricular and His-Purkinje (HPS) signals obtained from the apical region of the LV apex and from the anterior LV basal region during ventricular fibrillation.

FIGS. 4A-4D are graphs showing example correlations between ventricular (V) and His-Purkinje (HPS) signals during differing times of ventricular fibrillation.

FIG. 5 shows another example of an electroporation catheter system in accordance with some embodiments.

FIG. 6 shows the electroporation catheter system in use in a heart of a patient.

FIG. 7 shows another example electroporation catheter system in use in a heart of a patient.

FIG. 8 shows another example electroporation catheter system in use in a heart of a patient.

Like reference numbers represent corresponding parts throughout.

### DETAILED DESCRIPTION

This document describes methods and materials for treating ventricular fibrillation. For example, this document describes methods and devices for delivering pulsed-electric field electroporation for ablation in a selective and non-thermal means that has high specificity for tissue destruction while minimizing collateral damage.

Ventricular fibrillation is the most common terminal arrhythmia in humans. Electroporation is a technique that uses rapid and very short bursts of high voltages of DC current to non-thermally introduce multiple nanopores with the cells' walls of surrounding tissue. The His-Purkinje system (HPS) has been implicated in the genesis of ventricular fibrillation. Ablation of focal Purkinje triggers can successfully treat arrhythmias, which can be achieved using electroporation. This specialized Purkinje tissue may also be responsible for maintenance of VF, and thus its modulation and/or tissue destruction may serve as treatment for VF. The methods and devices provided herein can ablate focal or widespread segments of specific and critical regions of Purkinje tissue with minimal damage to healthy heart tissue which can be reversible and used for electroporation-mapping, modulation of Purkinje signals to evaluate effect, utilized to temporarily break the arrhythmia, or irreversible with complete destruction of Purkinje tissue as a means to treat VF.

One of the major reasons the problem remains unsolved is that there is no cross-over ability from the basic to the clinical realm. VF has been much better studied in cell or tissue preparations, such as Langendorff studies, where the tissue is ex vivo and artificially perfused. The hemodynamics as well as the autonomic system is not similar to that in vivo, and thus such findings may not be relatable to human VF. In clinical scenarios, due to VF being a lethal rhythm, there is immediate shock therapy delivered and drugs, and thus is short-lived and not well studied.

In order to do this, the inventors have endeavored to be able to both map the critical aspects during ventricular fibrillation and ablate these regions in order to eliminate and “cure” the patient. This requires the solution of being able to understand ventricular fibrillation (e.g., to map it and doing so in a meaningful way to impact it) and eliminate it (e.g., ablate the critical components to destroy the tissue responsible for VF).

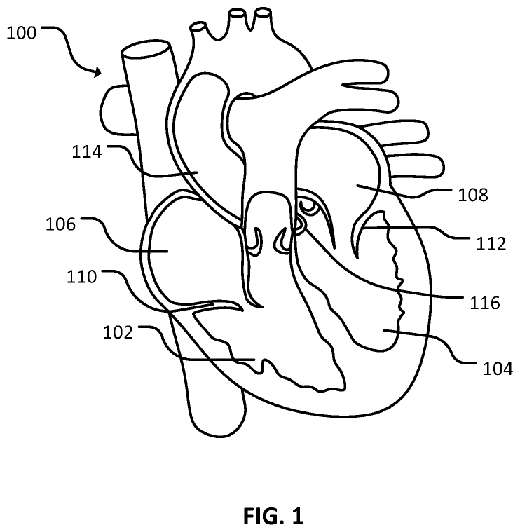

Referring to FIG. 1, a heart 100 includes a right ventricle 102, a left ventricle 104, a right atrium 106, and a left atrium 108. A tricuspid valve 110 is located between right atrium 108 and right ventricle 102. A mitral valve 112 is located between left atrium 108 and left ventricle 104. A semilunar valve 116 is located between left ventricle 104 and aorta 116. Right ventricle 102 and/or left ventricle 104 can include Purkinje tissue. Purkinje fibers can be located in the inner ventricular walls of the heart and are specialized conducting fibers that allow the heart's conductive system to create synchronized contractions to maintain a consistent heart rhythm. Purkinje fibers can be superficial in right ventricle 102 and/or left ventricle 104. In some cases, there can be millions of Purkinje fibers. Purkinje fibers can also initiate tachyarrhythmias, such as those that cause ventricular fibrillation. These tissues may also be critical in maintenance of VF. Thus, modulation and/or ablation of critical segments of this tissue may render a person free of VF inducibility or result in an increase in a VF threshold for sustaining/maintaining this rhythm.

As described further below; devices and methods for administering electroporation to locations of the heart 100 such as, but not limited to, the right ventricle 102 or the left ventricle 104 are provided herein. In some embodiments, bipolar electroporation can be delivered endocardially and/or epicardially. Moreover, using the provided devices and methods for administering electroporation, the Purkinje fibers can be targeted. Additionally, the ventricular myocardium can be targeted in the heart, such as the moderator band, right and left papillary muscles, the right and left septum of the ventricle, false tendons, etc. Furthermore, both Purkinje and ventricular tissue can both be targeted to have the desired effect of destroying tissue to eliminate VF and/or render a heart unable to go back into VF. In some cases, using the provided devices and methods, a catheter (e.g., a balloon catheter) can be used as part of a system for hemodynamic support during ablation. Using such devices and techniques, treatment of ventricular fibrillation can be achieved.

### A Novel Experiment of Long Duration Ventricular Fibrillation to Determine Regional and Temporal Variations of Ventricular and Conduction Tissue Activity

As stated above, ventricular fibrillation is a lethal rhythm that has received limited rigorous study in humans and large animal models. The critical structures necessary for VF maintenance are poorly understood and longer-term study of this rhythm may provide mechanistic insight. Accordingly, the inventors performed an experimental study which included complete endocardial and epicardial mapping during long duration VF in intact canines to evaluate chamber and time specific changes in ventricular and conduction system tissue.

Because the identification of discrete regions within the HPS that could serve as ablation targets would be clinically relevant, the inventors performed experimental systematic biventricular endocardial and epicardial mapping of long duration VF in canines using a multi-electrode catheter and an electro-anatomic mapping system, and sought to determine sites of fastest local ventricular and conduction system activity, as well as those with the highest regularity across both endocardial chambers and epicardium to determine critical elements necessary for VF maintenance.

### Experiment Methods

VF was induced and mapped in four canines. After VF induction, diagnostic mapping catheters were positioned in the left ventricle (LV), right ventricle (RV) and epicardial space. Endocardial and epicardial mapping was performed in sequence within a period of 15-minute intervals—LV endocardium was mapped first, followed by the RV, and then the epicardial surface (approximately 5 minutes per chamber was permitted). The left ventricle (LV), right ventricle (RV), and epicardium were mapped in sequential fashion during sinus rhythm and during 0-15 min, 15-30 min, 30-45 min, and 45-60 min after VF induction. Signals were further classified from each chamber into 3 or 4 regions as described below. Band pass filter settings were 0.5-1,000 Hz and the notch filter was programmed off to optimize for the recording of low amplitude and high frequency ventricular and HPS electrograms.

Ten consecutive beat tracings were analyzed to determine average cycle length (CL) and regularity of ventricular and His-Purkinje system (HPS) signals during each time period. Regularity, defined as a regularity index (RI), is the percentage of signals with CL within one standard deviation from the mean for a particular cardiac region and time.

The CL and maximum amplitude were measured from at least one set of the ten consecutive ventricular electrograms obtained from each of 4 LV regions, 3 RV regions, and 3 epicardial regions (described below). CL was measured from the peak of each ventricular signal to the peak of next ventricular signal. Conduction system signals were registered as sharp high frequency potentials prior to ventricular electrograms. Proximal conduction system signals including His and left or right fascicular signals were determined as previously measured where the ventricular signal was separated by a short isoelectric period whereas distal conduction (Purkinje) had no isoelectric period. Amplitude was measured from the peak or trough of the ventricular signal to baseline, whichever was higher. Regularity (RI) was assessed.

### Experiment Results

Across the four canine experiments, a range of VF duration from 45 to 73 min was observed. During VF, the fastest ventricular CL was present in the RV apical region (apical region includes ventricular muscle and papillary muscles from here on in), (70±10 msec) at 0-15 min, RV apical region (89±31 msec) at 15-30 min, anterior epicardial RV (80±3 msec) at 30-45 min and LV apical region (242±163 msec) at 45-60 min. The fastest CL between HPS signals was identified in the RV apical region (77±17 msec) at 0-15 min, RV inflow and free wall (89±12 msec) at 15-30 min, LV apical region (83±14 msec) at 30-45 min and inferior and inferolateral LV (145±23 msec) at 45-60 min. Regularity of the ventricular signals were highest in the RV inflow and free wall (RI 78%) at 0-15 min, RV apex (RI 86%) at 15-30 min, LV septum and epicardial anterior RV (RI 80%) at 30-45 min, and anterior and anterolateral LV (RI 75%) at 45-60 min. Regularity of the HPS signals were highest in the RV apical region (RI 80) at 0-15 min, LV apical region (RI 80) at 15-30 min and inferior and inferolateral LV (RI 83) at 30-45 min. The ventricular and HPS signals were dissociated during 0-45 min of VF but seemingly associated during 45-60 min with a correlation coefficient (R) 0.863.

Both ventricular and HPS CLs were fastest and had the highest regularity in the distal RV during the initial periods of VF and in the LV during longer duration VF.

The characteristics of ventricular and HPS signals classified by cardiac region and timeframe are presented immediately below in Table 1.

TABLE 1
Cycle length, voltage and regularity of ventricular (V) and
His-Purkinje (HPS) signals during ventricular fibrillation.

| | Time | V | CL (V, | CL (HPS, | Voltage | RI | RI |
|---|---|---|---|---|---|---|---|
| Location | (min) | count | msec) | msec) | (mV) | (V) | (HPS) |
| Anterior and | 0-15 | 60 | 101 ± 20 | 83 ± 19 | 11 ± 5 | 60 | 75 |
| anterolateral | 15-30 | 50 | 105 ± 24 | 108 ± 23 | 4 ± 1 | 74 | 77 |
| LV | 30-45 | 40 | 153 ± 79 | 149 ± 65 | 6 ± 3 | 71 | 70 |
| | 45-60 | 6 | 604 ± 213 | 343 ± 119 | 3 ± 0 | 75 | 83 |
| Inferior and | 0-15 | 60 | 100 ± 16 | NA | 15 ± 4 | 67 | NA |
| inferolateral | 15-30 | 40 | 107 ± 24 | 95 ± 2 | 5 ± 1 | 70 | 70 |
| LV | 30-45 | 38 | 142 ± 75 | 106 ± 32 | 4 ± 2 | 68 | 83 |
| | 45-60 | 22 | 309 ± 334 | 145 ± 23 | 4 ± 1 | 73 | 70 |
| LV apical | 0-15 | 70 | 98 ± 18 | NA | 12 ± 7 | 67 | NA |
| region | 15-30 | 40 | 92 ± 20 | 104 ± 34 | 5 ± 2 | 68 | 80 |
| | 30-45 | 36 | 136 ± 111 | 83 ± 14 | 3 ± 1 | 78 | 70 |
| | 45-60 | 23 | 242 ± 163 | 154 ± 31 | 4 ± 1 | 74 | 80 |
| LV septum | 0-15 | 60 | 104 ± 20 | 111 ± 20 | 9 ± 2 | 77 | 60 |
| | 15-30 | 50 | 108 ± 31 | 104 ± 32 | 6 ± 2 | 70 | 78 |
| | 30-45 | 40 | 139 ± 83 | 129 ± 43 | 3 ± 2 | 80 | 67 |
| | 45-60 | 24 | 250 ± 150 | 189 ± 60 | 6 ± 5 | 71 | 70 |
| RV apical | 0-15 | 50 | 70 ± 10 | 77 ± 17 | 10 ± 4 | 74 | 80 |
| region | 15-30 | 50 | 89 ± 31 | NA | 4 ± 1 | 86 | NA |
| | 30-45 | 23 | 202 ± 185 | 139 ± 58 | 4 ± 3 | 65 | 70 |
| RV inflow | 0-15 | 40 | 74 ± 10 | NA | 6 ± 2 | 78 | NA |
| and free | 15-30 | 40 | 97 ± 21 | 90 ± 12 | 6 ± 1 | 73 | 70 |
| wall | | | | | | | |
| RVOT | 0-15 | 50 | 85 ± 28 | 75 ± 3 | 5 ± 3 | 74 | 66 |
| | 15-30 | 40 | 93 ± 10 | 91 ± 2 | 4 ± 2 | 73 | 70 |
| | 30-45 | 30 | 162 ± 58 | 137 ± 96 | 4 ± 2 | 68 | 75 |
| Epicardial - | 0-15 | 50 | 77 ± 105 | NA | 12 ± 3 | 67 | NA |
| anterior RV | 15-30 | 45 | 160 ± 130 | NA | 8 ± 11 | 69 | NA |
| | 30-45 | 20 | 79 ± 3 | NA | 7 ± 0 | 80 | NA |
| Epicardial - | 0-15 | 40 | 136 ± 34 | NA | 18 ± 7 | 70 | NA |
| anterior and | 15-30 | 18 | 163 ± 85 | NA | 2 ± 0 | 67 | NA |
| lateral LV | | | | | | | |
| Epicardial - | 0-15 | 47 | 132 ± 83 | NA | 10 ± 3 | 72 | NA |
| posterior RV | 15-30 | 26 | 146 ± 120 | NA | 7 ± 4 | 73 | NA |
| and LV | | | | | | | |

The regional differences of regularity during VF are presented in Table 1. The ventricular signals were most regular in the RV inflow and free wall (RI 78%) at 0-15 min, RV apical region (RI 86%) at 15-30 min, LV septum and epicardial anterior RV (RI 80%) at 30-45 min and anterior and anterolateral LV (RI 75%) at 45-60 min. The HPS signals were most regular in the RV apical region (RI 80%) at 0-15 min, LV apical region (RI 80%) at 15-30 min and inferior and inferolateral LV (RI 83%) at 30-45 min (Table 1).

All four dogs had normal voltages during sinus rhythm. Average voltage from all regions was 7.3±0.9 mV. The amplitude of ventricular signals gradually decreased with time (Table 1). Throughout VF mapping, the EAM chamber volume gradually decreased for all chambers.

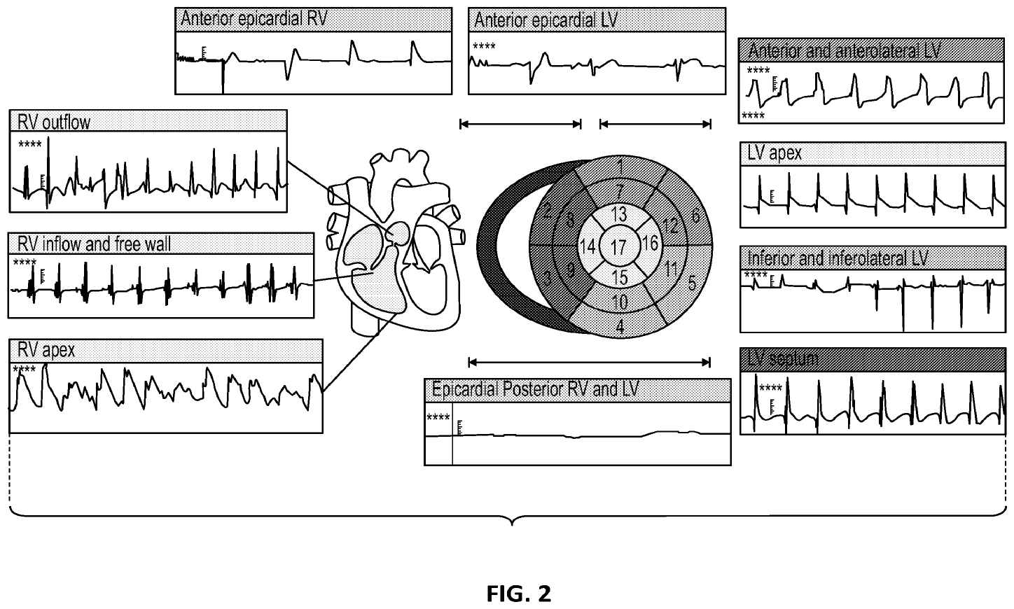

As illustrated in FIG. 2, there was significant regional variability during all stages of VF. As VF persisted, intracardiac activity became slower and more organized. The endocardium demonstrated sustained electrical activity despite electrical silence on the epicardium and the surface electrocardiogram.

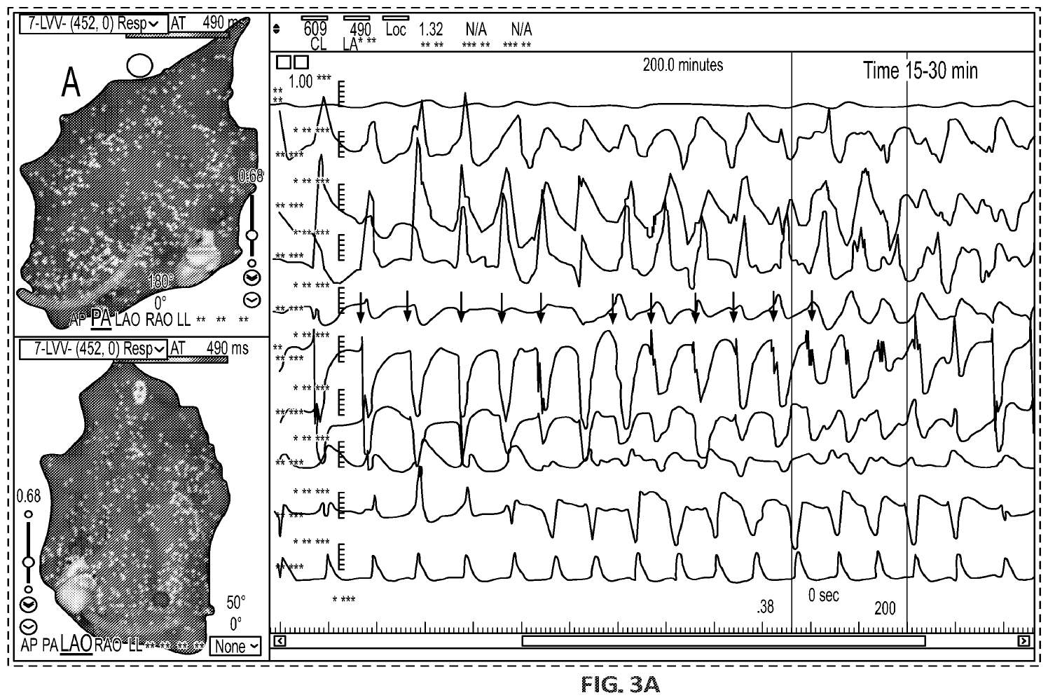

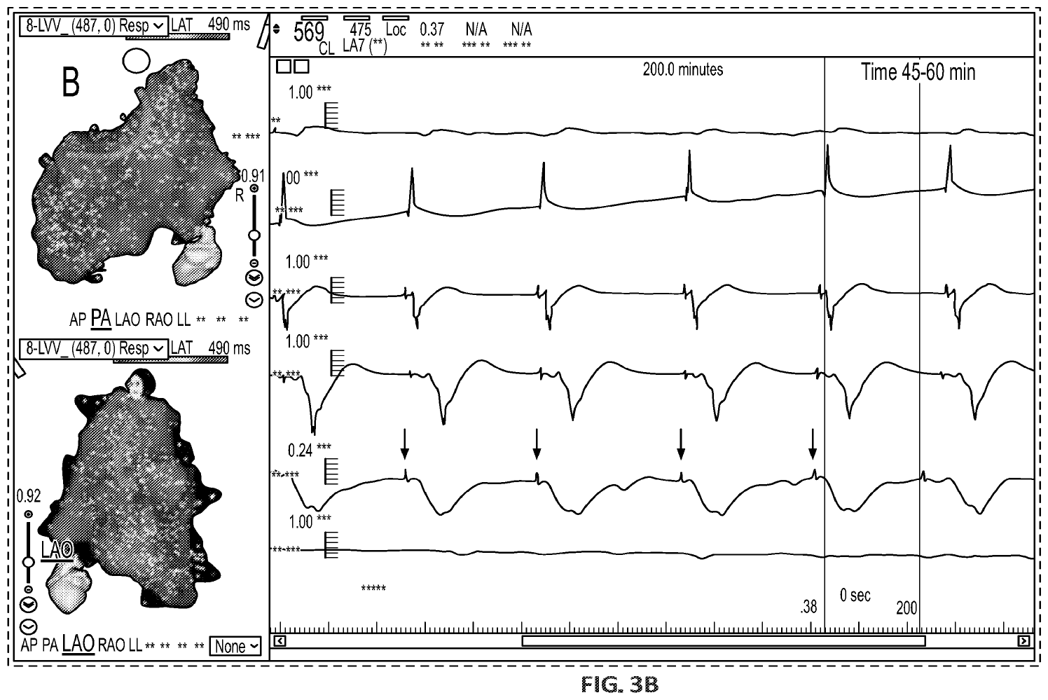

As illustrated in FIGS. 3A and 3B, HPS signals were intermittently discernible during early stages of VF but became more evident during later stages of VF with organization of the ventricular signals. In FIGS. 3A and 3B, the white arrows denote HPS signals.

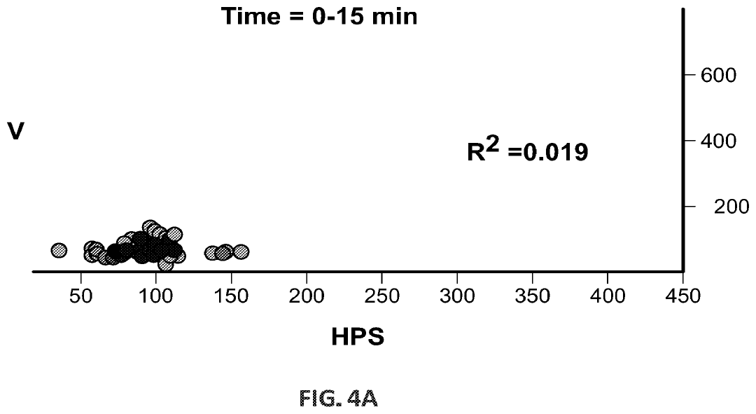

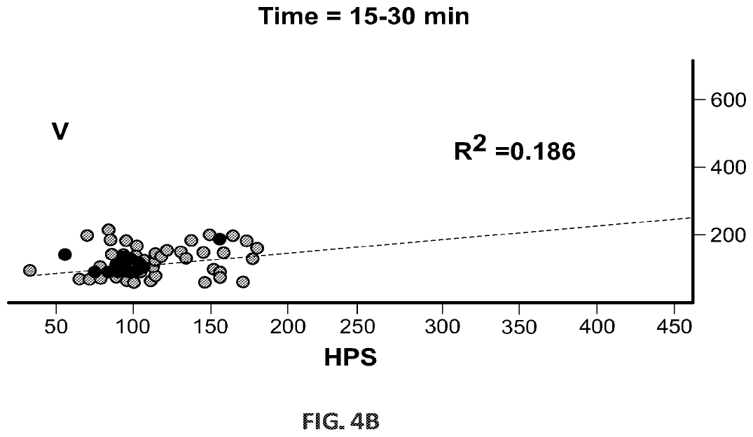

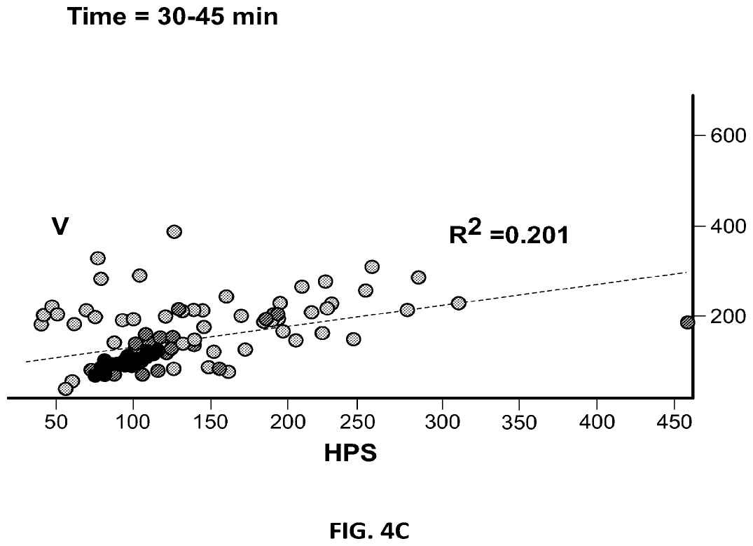

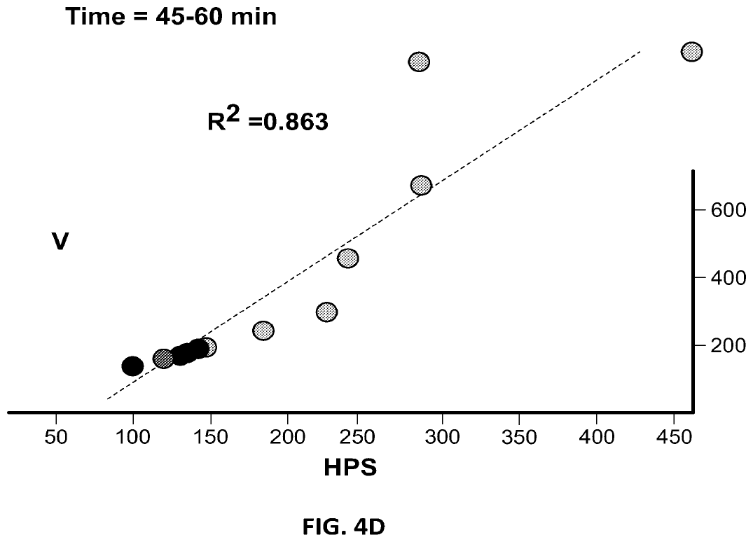

As illustrated in FIGS. 4A-4D, there was no association between ventricular and HPS electrograms between 0 and 45 min of VF, however there was seemingly 1:1 association in the later stages of VF from 45 to 60 min (R coefficient 0.863). At the end of VF, the last regions to demonstrate ventricular activity were the inferior and inferolateral LV at 45-60 min in three dogs and the RV outflow tract at 30-45 min in one dog. The last HPS signals were mapped in the LV septum at 45-60 min, the anterior and anterolateral LV at 45-60 min, the inferior and inferolateral LV at 45-60 min and inferior and inferolateral LV at 30-45 min in each of the dogs, respectively.

### CL Variability Throughout VF

The regional differences of CL during VF are presented in Table 1 above. The average CL was 121±5 msec for all ventricular signals, and 119±6 for all HPS signals. The fastest ventricular CL was present in the RV apical region (70±10 msec) at 0-15 min, RV apical region (89±31 msec) at 15-30 min, anterior epicardial RV (80±3 msec) at 30-45 min and LV apex (242±163 msec) at 45-60 min. The fastest HPS signals were identified in the RV apical region (77±17 msec) at 0-15 min, RV inflow and free wall (89±12 msec) at 15-30 min, LV apical region (83±14 msec) at 30-45 min and inferior and inferolateral LV (145±23 msec) at 45-60 min.

### Discussion

Some of the salient findings of this experimental study include: 1) the fastest ventricular CL and highest regularity during VF was present in the RV endocardium during the earlier stages of VF (0-30 min), 2) the fastest ventricular CL and highest regularity during VF was present in the LV endocardium during the later stages of VF (30-60 min), and 3) the HPS and ventricular signals seemed to be dissociated during early VF but frequently demonstrated 1:1 association during late VF.

Developing a cure for VF is a daunting task mainly because of its chaotic electrical appearance that makes it hard to identify focal drivers as well as region necessary for VF maintenance. Currently ablation of idiopathic VF targets focal initiating triggers including premature ventricular complexes. However, determining heart regions that sustain (rather than trigger) VF and that are amenable to ablation has been a long-term goal of prior investigators. Prior research supports the potential of the HPS involvement for VF maintenance. This has been demonstrated in a three-dimensional computerized heart model in which the Purkinje-muscle junction was responsible for maintaining reentry with subsequent passive spread to the intramyocardial and epicardial layers. Chemical ablation of the HPS in hearts perfused with Lugol's iodine solution altered duration of VF compared to controls. Additionally, catheter ablation of critical Purkinje tissue, either with radiofrequency or most recently electroporation, delivered through basket catheters decreased VF recurrence and vulnerability to VF. Our study supports these prior findings by 1) demonstrating that the distal RV and LV (which house the distal conduction system) are the latest to lose electrical activity with persistent long duration and 2) showing HPS signals preceding ventricular signals in the later stages of VF when electrical activity organizes (FIGS. 3A and 3B).

The three dimensional anatomic and electrophysiologic complexity of the HPS pose significant challenges for ablation and/or to map during VF. Further characterization of specific regions of interest is necessary to develop a ventricular lesion set that could be applied to decrease VF burden in experimental models. Specific regions of interest further supported by our study are the papillary muscles, distal trabeculated RV or LV regions and, in some cases, the RV outflow region. Given the small size of the RV in canines, the distance between the base of the right papillary muscles and true apex is smaller than humans. As a result, the fastest and most regular VF activity in the RV apex may have reflected papillary muscle activity. The role of the RV in the genesis and maintenance of VF has likely been underemphasized in the literature since many VF studies performed mapping of the LV only. However, the RV has at least comparable trabeculation volume as the LV and can thus provide numerous anatomic pathways for reentry. In addition, regions such as the RV outflow and moderator band are well known to be arrhythmogenic and may require ablation to decrease VF burden. Based on these observations and preliminary findings, substrate VF ablation consisting of an empiric lesion set to endocardially transect distal Purkinje tissue with particular emphasis on arrhythmogenic structures such as the papillary muscles or the RV outflow tract can be suggested. Alternatively, targeted ablation of localized reentrant foci after VF mapping can be entertained. Recently, areas of functional reentry in patients with refractory VF and multiple defibrillator shocks was successfully mapped and ablated.

### Experiment Conclusions

The experiments confirmed the presence of significant regional and temporal variability of ventricular fibrillation and suggests that certain regions may have higher arrhythmogenic potential. The experiments also revealed that the regions of fastest CL and highest regularity of ventricular and HPS electrograms were noted to be in the distal RV during early VF and in the LV endocardium during late VF.

In view of the results of the above experimental study performed by the inventors, the following novel devices, delivery systems, and protocols in order to realize the potential of curing ventricular fibrillation have been developed.

### Example Device and Method Embodiments

Balloon-based catheters with one or more electrodes that can deliver monopolar or bipolar electroporation energy for treating VF have been developed by the inventors. The one or more electrodes can be on the surface of the balloon in some embodiments. The one or more electrodes can be in an interior of the balloon, and/or on a catheter shaft in some embodiments. In some embodiments, a combination of electrodes on the surface of the balloon and/or in an interior of the balloon, and/or on a catheter shaft can be included.

Such balloon-based catheters can include one or more of the following features (in any combination). For example, in some embodiments a control system is included so that the balloon inflates/deflates based on gating, or synchronization, with the patient's ECG (e.g., to coordinate with ventricular contraction).

In some embodiments, a control system is included so that the balloon inflates/deflates based on a rate of change of pressure in the left ventricular cavity of the patient.

In certain embodiments, a control system is included so that the balloon inflates/deflates based on a timing cycle.

In particular embodiments, a control system is included so that the balloon inflates/deflates based on impedance changes in the heart based on contraction of the heart and/or tissue proximity.

In some embodiments, a control system is included so that the balloon inflates/deflates based on impedance changes of the target tissue, e.g., so as to automatically shut-off when ablation is completed.

In some embodiments, a control system is included so that the balloon expansion can be continued until a certain surface tension (or balloon inflation pressure) is reached in order to ensure adequate tissue contact by the balloon, and/or the balloon inflation can be timed in order to inflate for a period and deflate for a specified time after in order to allow for adequate perfusion of the heart chamber and maintenance of blood pressure.

In some embodiments, the expansion of the balloon is tied to a pressurized inner balloon with pores in order to inflate to a certain amount as desired.

Further, in some example embodiments a control system is included so that the balloon inflation and expansion is coordinated with the patient's systole/diastole in order to augment ventricular output to add safety to the procedure.

In particular embodiments, the material of the balloon is porous in order to not constrict or impede blood flow in the chamber that would otherwise lead to hemodynamic instability.

In some embodiments, the balloon-based catheter can comprise or be connected to a suction port that draws blood from the ventricle and discharges the blood in the distal aorta to perform a catheter-based bypass system. Not only would such a system provide hemodynamic stability; but also catheter stability by limiting blood volume displacement of the catheter.

The inventors have also developed devices and methods that target cardiac ganglia in addition to the ventricular myocardium and Purkinje tissue. In some embodiments, this includes delivering bipolar ablation from the ventricular endocardium, mid-myocardium, and/or epicardium.

Additionally, or alternatively, this can include delivering bipolar ablation or electroporation with the electrodes in the endocardium, on both sides of the septum, in the endocardium and epicardium, and/or both in the pericardial space. One of the goals of such a method is to destroy the ventricular cardiac ganglia and spare ventricular myocardium as desired, and vice versa.

The inventors have also conceived of certain bipolar ablation catheter constructions and alternate energy delivery sites, such as the stellate ganglia. In addition to the ablation/electroporation of the ventricular myocardium, Purkinje tissue, and cardiac ganglia, pulsed-electric field electroporation ablation in combination with modulating the stellate ganglion either reversibly or irreversibly can be delivered to either the right stellate ganglion, the left stellate ganglion, or both. This can also provide an additional anti-fibrillatory effect. This could be done percutaneously via a right or left internal jugular approach, as well as via thorascopically. In some embodiments, this includes a plurality of combined procedures as noted above in order to give the best change of curing VF—endocardial/epicardial/pericardial, stellate ganglion, and/or combinations thereof.

The inventors have also conceived of certain bipolar ablation catheter constructions and alternate extracardiac structures that can also serve as additional vantage points not reachable other than with open chest surgery. In some embodiments, this would include a catheter in the endocardial space as well as the epicardial space. Furthermore, in some embodiments this would include an additional catheter from a vantage point such as the arch of the aorta to serve as a return electrode for bipolar ablation. One embodiment includes a string like catheter over a small balloon/wire to track around the great arteries and thus providing leverage to access the associated ganglia. These are difficult to get to even surgically and thus a percutaneous approach would have significant benefit. In some embodiments, this would also include a catheter sitting in the pericardial sac and a return electrode mesh seated in the oblique and/or transverse sinus in order to selectivity ablate/modulate the structures in between. In particular embodiments, this would include a horseshoe-like catheter placed around the pericardial space in order to mirror any location inside the ventricular cavity. These could also be used in combination with a catheter as noted above which could track along the great arteries to provide varied vantage points to optimize multi-site ganglia destruction.

The inventors have also conceived of certain bipolar ablation catheter constructions and alternate mediastinal structures using veins, arteries, and/or the trachea/bronchus for additional vantage points to module cardiac and neural tissues. In some embodiments, this includes a pulmonary artery catheter designed as one electrode return limb to complete bipolar ablation. In some such embodiments, the additional bipolar electrode catheter could be placed in the superior mediastinum. This can also include the combination of the right pulmonary artery and the posterior wall/roof. In some embodiments, this can include the use of the azygous vein as the posterior return electrode as well. In particular embodiments, this can include the use of the combination of the aorta to left mainstem bronchus. In some examples, this can include the use of the combination of the right mainstem bronchus and azygous vein. In some embodiments, this can include the use of the combination of the superior intercostal vein (anterior and with the vagus). Any and all of the above-mentioned combinations could also be ablated in order to treat hypertension or stimulate for anti-syncope treatment.

The inventors have also conceived of the utilization of the trachea/endotracheal tube as a vantage point for a set of electrodes to complete bipolar ablation. In some embodiments, this would utilize the trachea as a posterior boundary when utilizing the endocardial, epicardial, pericardial, aorta or pulmonary artery and thus adding a posterior boundary. This could also include using a breathing particle in the lungs in gaseous form that is able to treat selectively bronchial tissue as a treatment for asthma.

Any and all of the embodiments described herein can be used in conjunction with drug delivery agents to improve/facilitate drug delivery and/or uptake.

The inventors have also conceived of new way of pace termination for treating ventricular fibrillation. Currently ATP (anti-tachycardia pacing) is used to pace-terminate patients out of ventricular tachycardia in ICDs. This is not of utility using endocardial or any type of leads, as the VF circuit can currently not be pace-terminated with current settings and tools available, given the inability to disrupt a critical part of the electrical circuit of this rhythm, as well as be invoked at a certain timing or after certain duration of VF. VF immediately triggers the ICD to shock given its lethality The inventors' experimental findings of regularity in certain regions of the heart, and at certain time points of ventricular fibrillation, indicate there are areas of regularity that lend themselves available for ATP to terminate VF as opposed to shocking. This can include any combination of an endocardial pacemaker and an epicardial pacemaker. This would include various combinations of recording as well as pacing from endo, mid, or epicardium in order to pace into the “regularized” portion of ventricular fibrillation to which a critical portion of the circuit can be timed and thus terminate the rhythm with ATP as could be done currently for ventricular tachycardia and ICDs. In some embodiments, this also includes a means of pacing a critical mass of epicardial tissue in order to “push” or “constrain” the VF circuit to the endocardial layers of the heart so that the critical aspects of the heart are manifest for mapping and ablation. Although the Purkinje/endocardial aspects of the ventricle have the strongest likelihood of responsibility in the persistence of the VF rhythm, it is unknown how much of the mid-epicardial tissue is required for maintenance. Thus, this permits a unique vantage point for “ATP for VF” by constraining the necessary elements of the component to where a device lead can pace and break this arrhythmia without the need for ICD shocks. This would provide a “painless” approach to treatment of VF as an ICD shock would not be required.

The inventors have also conceived of novel vantage points for defibrillation and/or pacemaker coils/leads for pace/shock termination of VF. An important aspect of VF initiation and maintenance is within the His-Purkinje system. There is a rich layering of this along the right and left ventricular septum as well as across and into the right and left ventricular papillary muscles. There is also HPS tissue within the moderator band as well as false tendons in the ventricle. Thus, the inventors have conceived of placing a screw-in coil longitudinally into the interventricular septum. This would permit detection of VF, as well as an intraventricular means of VF termination with pacing, brief pulses of electroporation for termination, and so on. This would also include non-traditional places for electrode deployment and placement. Such non-traditional places for electrode deployment and placement can include, but are not limited to: (i) distal coronary artery deployment (such as in the septal perforator), (ii) one or more electrode screws in the distal coronary venous branches that could serve as vantage points for energy delivery including electroporation energy delivery (including to the mid-myocardium), and (iii) a screw placed directly to the papillary muscle head into order to have a direct and deep stable route to the papillary muscle and Purkinje network.

In some cases, using the catheters provided herein, various drugs can be delivered locally. For example, the drug can be delivered to the Purkinje conduction tissue in the ventricle, where a focus of pro-arrhythmic activity can be disrupted.

In some cases, the drug can be administered in addition to delivering electroporation to the ventricle. Electroporation can alter the cellular lipid bilayer by providing an electrical or magnetic field to the tissue site of interested, while the drug is being administered locally. In some cases, a pulsed DC electroporation current can be used. In some cases, the drug can be administered simultaneously with the delivery of electroporation. Such a combination of electroporation and drug delivery can promote the update of the drug into the cell, and thus add to the safety and specificity profile of the drug. For example, electroporation can open the cell membrane, the drug can be administered and taken into the cell, and then electroporation can be used to seal the cell membrane. In some cases, the drug can be administered in isotonic saline with albumin (e.g., 5% albumin), to allow protein-drug binding and provide a lipophilic partitioning to favor membrane portioning to alter bilayer proteins to create changes in transmembrane potential.

In some cases, to target delivery of drugs to specific tissue (e.g., Purkinje tissue), in addition to administering the drug locally, the electroporation can be modified to facilitate uptake of the drug by the specific tissue. For example, parameters (e.g., duration, timing, number, and/or amplitude) of pulsed-electric fields to incite electroporation can be modulated. In some cases, parameters of electroporation can be selected such that reversible electroporation is delivered. In some cases, reversible electroporation for Purkinje cells can allow the cells to “open” and “close,” and thus capturing the drug inside the cells. In some cases, inversions of pulse polarity of electroporation can be used to leverage the specificity of drug delivery and retention in tissues of interest. In some cases, parameters of electroporation can be used to preferentially open membranes for enhanced selectivity of tissue (e.g., Purkinje uptake and not ventricular myocardial uptake).

In some cases, electroporation could be used solely as a means to increase uptake of drugs into cells, which can increase safety and selectivity of the procedure. In some cases, the electroporation can allow for uptake and retention in the cells and molecular ablation can take place. In some cases, reversible electroporation can be used to re-seal the cell. In some cases, irreversible electroporation can be delivered after reversible electroporation.

In some embodiments, a distal portion (e.g., a tip) of the catheter can include a sensor. In some cases, the sensor can detect an extracellular concentration of sodium, potassium, calcium, pH, etc. In some cases, the sensor can be in communication with a feedback circuit, such that electroporation is ceased when the cellular expulsion into the local extracellular milieu is complete (as detected by the sensor).

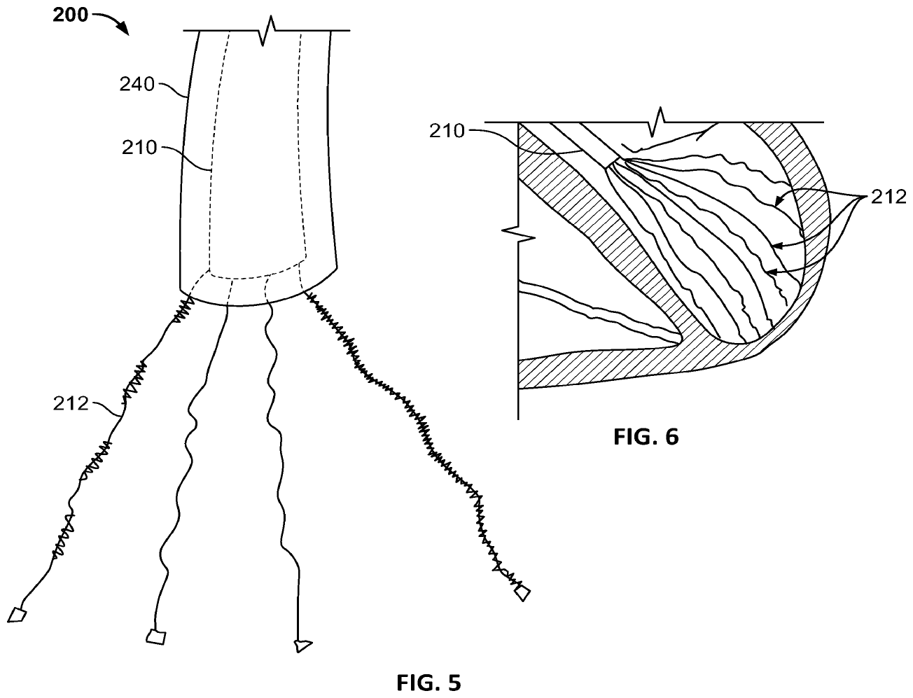

As shown in FIGS. 5 and 6, in some embodiments an electroporation catheter system 200 includes a delivery sheath catheter 240 and an electroporation catheter 210 that is slidably disposed in the delivery sheath catheter 24. In some embodiments, the delivery sheath catheter 240 is constructed of a material or is coated partially or in full with material such as Mu-Metal®. In some embodiments, the delivery sheath catheter 240 serves as a large return electrode (in conjunction with the electroporation catheter 210).

The electroporation catheter 210 is configured for use to perform cardiac sensing, pacing, ablation, electroporation, take sensor measurements (e.g., temperature using thermistors, pH measurements, etc.), and combinations thereof. The electroporation catheter 210 can define a lumen so that the electroporation catheter 210 can be advanced over a guidewire (e.g., a long J-tipped wire to use as a safe rail to advance the electroporation catheter system 200 through the mitral valve and into the ventricle). In addition, in some embodiments a small extendible guide catheter with a J tip can be used with steerability from the proximal handle. This will allow for safe crossing and maneuverability. In addition, this can also provide additional stability on structures such as the septum and papillary muscles that can be tough to maintain contact on with the beating heart.

In some embodiments, the electroporation catheter 210 can have irrigation ports for inflow of solutions. The electroporation catheter 210 can be used to treat (e.g., ablate and/or electroporate) Purkinje conduction tissue, or myocardium tissue, and/or both Purkinje conduction tissue and myocardium tissue.

The electroporation catheter 210 includes one or more flexible elements 212 (e.g., varying from 3-10 flexible elements 212 that can “fan out”). The extent of the fanning out of the one or more flexible elements 212 is controllable by the position of the electroporation catheter 210 relative to the delivery sheath catheter 240. That is, the one or more flexible elements 212 can be allowed to fan out in a wider pattern by moving the electroporation catheter 210 distally relative to the delivery sheath catheter 240. Moreover, in some embodiments the one or more flexible elements 212 can be individually extended from the electroporation catheter 210. In some embodiments, the electroporation catheter 210 includes a screw mechanism that can be used to center or pull the one or more flexible elements 212 down to the apex of the left ventricle.

Each electroporation catheter 210 of the one or more flexible elements 212 can be configured in a particular arrangement, and some of the one or more flexible elements 212 can be configured differently than any of the other flexible elements 212. For example, the flexible elements 212 can be configured as a long linear flexible single electrode, a multi-electrode flexible element, a ground electrode, and so on. In some embodiments, each of the one or more flexible elements 212 is electrically wired independently of the others. Each of one or more flexible elements 212 has the ability to be used as a wire/nitinol extension to which electrodes can be placed on top of/on the outside of. In addition, the one or more flexible elements 212 can also be connected with a wire to which the electrodes are run through in parallel. In one embodiment, the splines of the catheter can be arranged in any combination of alternating electrode arrays. For example, one spline can serve to house a multitude of electrodes, with the neighboring spline can serve as a large, long electrode itself. This can be used in combination with several splines with alternation occurring in several combinations. In addition, the proximal to distal splines can have differential electrode spacing, coating/shielding, as well as positing in order to provide a selective means of targeted ablation. For example, the distal HPS can be targeted and the proximal HPS can be spared.

The electroporation catheter system 200 has the ability to access the right and left ventricle safely while negotiating the valve apparatus safely. Furthermore, large regions of ventricular and HPS tissue can be obtained rather quickly and with good contact. Finally; the design has the all-in-one ability for the device electrodes that can have the ability for conformational chamber design and can perform mapping, pacing, and electroporation ablation of ventricular myocardium and/or the HPS in order to address all the needs for success treatment delivery.

In some embodiments, the one or more flexible elements 212 can also be used to wrap around the base of the papillary muscles, as well as provide a means to get to the septal and lateral aspects of the ventricle. In some embodiments, a shielding agent such as Mumetal is used in order to provide a form of electric shielding to prevent ablation of certain regions of the heart such as the proximal portion of the ventricles and conduction system. The delivery sheath catheter 240 can have the ability for various flexibility curves (e.g., can be selectively deflectable) to permit bidirectional movement of the mapping/ablation catheter. There can be a multiplicity of the electrodes on one or more of the flexible elements 212 versus one long electrode. A combination of pacing and ablation can be varied and modulated based on desire of size of pacing and electric field and desired region for ablation. The electroporation catheter 210 can be long enough to extend from the base to the apex of the ventricle. As shown in FIG. 6, a return electrode for any of the electrodes of the flexible elements 212 (on the catheter splines and/or the sheath) can be placed in the epicardial space (to ablate across the LV wall) or in the RV (in order to ablate across the septum). The specificity of ventricular versus HPS ablation and vice versa can be modulated by varying the delivery of pulsed-electric fields from the electroporation catheter system 200 with a plurality of delivery protocols and parameters.

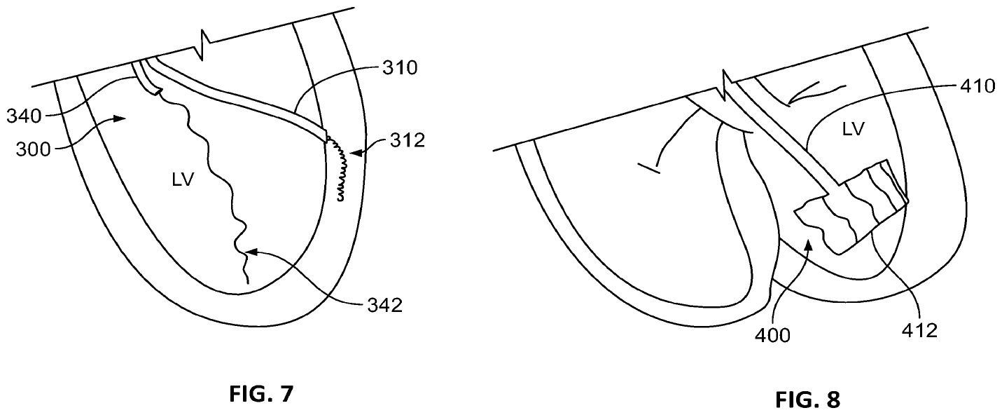

Referring to FIG. 7, another example electroporation catheter system 300 can be used to treat (e.g., ablate and/or electroporate) Purkinje conduction tissue, or myocardium tissue, and/or both Purkinje conduction tissue and myocardium tissue. In some cases the electroporation catheter system 300 can be used at the bedside, may not require the use of general anesthesia, and may provide painless defibrillation.

The electroporation catheter system 300 includes a first catheter 310 that includes a long helical coil return electrode 312. The electroporation catheter system 300 further includes a second catheter 340 that includes an internal floating electrode 342.

In some embodiments, the helical coil return electrode 312 acts as a catch point for the electric field from the internal floating electrode 342, allowing for ablations only on the endocardium. The internal floating electrode 342 can be a single electrode, or a multiple electrode device. In some embodiments, the internal floating electrode 342 can include a deployable helical coil for anchoring to tissue.

Referring to FIG. 8, another example electroporation catheter system 400 can be used to treat (e.g., ablate and/or electroporate) Purkinje conduction tissue, or myocardium tissue, and/or both Purkinje conduction tissue and myocardium tissue.

The electroporation catheter system 400 includes a catheter 410 and an electrode array 412. In some embodiments, the electrode array 412 can unroll when deployed from a delivery sheath catheter. In some embodiments, the electrode array 412 can unroll when a mechanism is activated. The electrode array 412 can unroll in any desired direction and manner. In some embodiments, the electrode array 412 comprises a mesh framework that includes interwoven electrodes in between, on-top of, or in a combination of on-top and within the framework. The floating electrode array 412 mesh will then unravel or unroll and be able to “drape” over large parts of the endocavitary region.

In some embodiments, the catheter 410 includes a steerable feature that is used to press the electrode array 412 up against the LV wall to provide additional stability and contact. For example, pushing the electrode array 412 up against the septal wall. Similarly, in some embodiments the electrode array 412 can be draped over a papillary muscle as well as the septum next to it. This electroporation catheter system 400 has all of the capabilities mentioned above with electrodes that can pace, map, and ablate via electroporation. This construct can also be used in combination with a second floating mesh placed in the pericardial space to allow for a bipolar ablation across the endo and epicardium. The second stabilizing catheter can be used to provide a return electrode. The stabilizing catheter can also be used to provide protection via mechanical and/or via shielding to permit the delivery of focused pulsed-electric fields.

The inventors have also conceived of a multi-faceted catheter with constructs that facilitate the ability to: (a) map and decipher His-Purkinje system (HPS) signals both in normal rhythm and during ventricular fibrillation, (b) deliver pulsed electric fields for electroporation ablation to selectively target the critical HPS tissue while minimizing ablation of healthy ventricular myocardium, and/or (c) provide access and stability to electroporate or ablate these structures which are widespread throughout the right ventricle, septum, and left ventricle.

In some embodiments, an example of a multi-faceted catheter for electroporation delivery includes a combination of a balloon and a snake-like, steerable inner-wire configuration. Given that the right ventricle (RV) and left ventricle (LV) endocardial regions have the His Purkinje System (HPS) draped across their surfaces, the intracavitary dimensions create a difficulty in obtaining stability for contact to ensure appropriate mapping and ablation of this tissue. In one embodiment, a catheter is deployed via transseptal access or from retrograde aortic access to get to the LV. The same can be from the inferior vena cava in order to access the RV. In some embodiments, the multi-faceted catheter has a spiral, snake-like shape that unwinds and curls via advancement of the catheter, and retraction from a handle to get the desired shape. For example, in some embodiments such a catheter can form an essentially circular ring via its form-fitting shape once exiting the catheter. This will permit movement along the septum of both the right and left ventricles, as well as around the papillary muscles of both the RV and LV. In addition, this essentially circular ring can go along the moderator band in the RV, as well as around, on top of, and underneath to be able to fully ablate the critical structure. In addition, this conformation can also have snake-like features so that the catheter can get in between areas of the mitral valve and sub-mitral valve apparatus that are in close approximation in the LV cavity. Such regions such as in between the papillary muscles, in between the papillary muscles and the septum, and in between false tendons.

The inventors have also conceived of a catheter with multiple, mini-tines that can retract and protract for stability. For example, one embodiment involves a catheter shaft with multiple mini-tines, which can be protruding such as mini-spikes in order to hook into any region in the LV (which is associated with the HPS). These flexible tines can provide catheter stability for mapping, localization, and stability for ablation with electroporation.

The inventors have also conceived of a catheter with a balloon-based exterior with an inner, flexible wire 2-4 Fr with torquability to provide stability in the ventricle between the papillary muscle, on the moderator band, or sub-mitral apparatus. Such a catheter can optionally include a side port for a small catheter to protrude and to be able to engage any aspect of the HPS and being able to pace, stimulate, and ablate tissue.

In addition, the inventors have conceived of using vantage points for bipolar electroporation via a combination of catheters with a cathode on one catheter and anode on the return electrode/catheter system. One such embodiment involves any of the catheters mentioned herein being utilized in the endocardium of the RV or LV or screwed into the septum. The return electrode for electroporation can be in the RV as a return. In some embodiments, this can be LV to RV and thus perform electroporation of the HPS along/in the septum. Another embodiment can have the catheter in the RV, LV, or septum, and the return electrode within the coronary sinus in order to perform both endocardial as well as epicardial ablation as desired.

The inventors have also conceived of novel electroporation devices, systems, and methods of the endocardial/epicardial tissue and/or HPS ablation using a novel vantage point in the Coronary sinus (CS). Another embodiment can be performed without entering the epicardial space and would have the catheter in the LV and an occlusive balloon inflated in the CS with a floating wire that would function as a return electrode for epicardial ablation as well as intramyocardial/mid-myocardial ablation via accessing venous tributaries with such a wire. In addition, with the occlusive balloon in the coronary sinus, a circulation of hypertonic saline or metal fragments can be induced along with the wire in place in order to provide a virtual return electrode for widespread ablation. In some embodiments, the reverse can be done by using such a vantage point from the CS electrode/wire as the cathode and any catheter as the return in order to perform epicardial ablation. The advantage being that with a more sub-selective and smaller space in which the bipole resides, the more the selective ablation that can be performed.

In some embodiments, the catheter and/or sheath can include a coating. One such embodiment includes a coating on the surface of both the sheath and catheter to serve as a return electrode and thus variable bipolar spacing for mapping and/or ablation. Another embodiment provides partial shielding (such as by using MuMetal/NuMetal for shielding or electrical insulation) of the catheter to limit and/or direct the area of the pulsed-electric field emitted from the catheter.

In order to map critical areas of the heart and neuro-system that are necessary for triggering and maintaining VF, in some cases hemodynamic support is needed (e.g., ECMO or cardiopulmonary bypass) in order to support the vital organs and provide blood circulation. This can be very difficult, expensive, require extensive expertise, resources, and has several large bore access sites and pumps required, as well as limited systems. A percutaneous means of providing hemodynamic support within a catheter sheath and system in order to provide circulatory support would provide a novel means of mapping and ablation and open the availability of more procedures to be performed safely. Accordingly, the inventors have conceived of the following additional devices, systems, and methods.

A device/system can be used for percutaneous access using a combination sheath with hemodynamic and catheter access capabilities to support left-sided circulation and access the left ventricle. One embodiment includes accessing the left ventricle (LV) via a retrograde aortic access approach with a double-barreled catheter (e.g., one sheath with two partitioned segments—one for hemodynamic support, one for catheter access for mapping and ablation). The distal portion of the sheath can reside in the LV, the left atrium (LA), or ascending aorta, which can either pump blood or remove blood from said space and via suction, or pump in order to transfer/deliver blood to the proximal port of the sheath (residing in the proximal ascending or descending aorta to supply rest of the body). In some cases, this flow path can also be reversed to go from the descending aorta to the sheath's distal port if it is desired to provide additional blood pressure support. Titration can be performed in order to minimize the size of the LV chamber and facilitate catheter contact. Another embodiment involves using the side port of a catheter as the distal portion of blood suction from the LV with deposition to the proximal portion via a catheter or sheath residing in the descending aorta. This can support the circulation with blood flow; oxygenation, saline, infusion, and/or a combination of pumping, peristalsis, and spinning from an outer pump/unit as part of the catheter system and apparatus. As an alternative to, or in addition to, the preceding, in some embodiments the LV can be accessed via a transapical approach, or a trans septal approach, e.g., from the right to the left atrium and then to the LV, transapical, or from the right ventricular with trans septal puncture to access the LV.

In another example embodiment, percutaneous venous access with a sheath can be performed in order to map and ablate in the RV. For example, in some embodiments this can be performed using a femoral vein, axillary vein, internal jugular vein. In such ways, access to the inferior vena cavae/superior vena cavae and right atrium can be achieved with a sheath with capabilities of hemodynamic support with proximal and distal ports. The distal port of the sheath can be advanced to the pulmonary artery and the proximal port would reside in RA or RV and thus could pump/suction blood to facilitate contact, mapping and/or ablation in the RV to access the RV papillary muscle, RV septum, His-Purkinje, and/or moderator band. This can also be used for right-sided VT ablation such as for ARVC or other cardiomyopathies.

In an additional example embodiment, a percutaneous cardiopulmonary bypass system using a combination of right-sided and left-sided access via sheaths in combination with an external pump circuit to oxygenate and pump blood can be used. In this embodiment, blood can be removed from the RA and RV and delivered to an external pump, and then delivered to the left-sided circulation after extracorporeal oxygenation. One embodiment would involve the use of venous access as noted above to remove blood from the right-sided circulation (e.g., from the RA, RV, or pulmonary artery). This would then be used in combination with any left-sided access to complete a bypass of the heart and lungs. For example, the removal of blood from the right-sided circulation can be delivered to the LA, LV, ascending or descending aorta via one or more sheaths in place at a plurality of various access points. The access points can include: A) via the left atrium from a trans-septal puncture from the right atrium; B) via retrograde access through the aorta with the sheath residing in the LV, ascending aorta, or proximal ascending aorta/descending aorta; C) a combination of connection of both sheaths to an exterior pump to facilitate oxygen exchange, filtration, and peristaltic pump can be performed; and/or D) the left-sided/arterial support can also be from an LVAD.

Another embodiment conceived of by the inventors includes a sheath with proximal and distal ports for blood entry/exit. The sheath and catheter include a port each, and one or both sheaths have a separate partition lumen for catheter access. The catheter itself would go through the sheath to be used for mapping and ablation. The catheter itself may or may not have a side port for suction/pumping of blood into/from the left-sided circulation. To provide complete veno-arterial bypass and full support, a second sheath can be placed from the right femoral vein and advanced into the inferior vena cava or right atrium to permit access to the right ventricle. This can also be advanced to the right ventricle and/or pulmonary artery. This can also be performed via right internal jugular access to get to the right atrium, right ventricle, or pulmonary artery via superior vena cava access and advanced to RV/PA. This can provide removal of blood from the right sided chamber/circulation and connected to a pump which is connected to the unit connected to the sheath for the left sided circulation deposition, or into the pulmonary artery to get to the lungs if desired. In some embodiments, the blood can be removed from the RA/RV (using the proximal port of the catheter) and dumped into the pulmonary artery (via the distal port of the catheter) in order to provide access to the RV for mapping and/or ablation, and also to provide blood flow continuous to the lungs and rest of the body.

In some embodiments, a bypass circuit of the left ventricular can be created using a trans-septal sheath across the RA-LA and advanced into the LV and ascending aorta. In such an embodiment, blood is taken from the left atrium and deposited via the distal port of the catheter in the ascending aorta. This would utilize over-the-wire guidance and a steerable two or four French catheter over-the-wire approach to get into the LV outflow tract. In some embodiments, a second trans-septal access would be used to access the LV from the LA in order to perform mapping and ablation.

In other embodiments, a bypass circuit can be established using a retrograde aortic access approach with a sheath/catheter delivery system to guide the catheter into the LA. In such an embodiment the catheter itself is used for steerability and maneuvering so that the sheath can be advanced into the LA, and the distal port of the sheath would remove blood from the LA. The proximal port on the sheath would be in the ascending aorta and would pump the blood to the rest of the body. In some embodiments, this would also include (if desired) a second partition so that a catheter can be used for access, removed, and positioned in the LV, and another portion of the sheath is used for hemodynamic support.

Another embodiment conceived of by the inventors involves one sheath that can be advanced across the ventricular septum from the RV to the LV, and on its way out deploy a device to close the gap. This can be utilized as a vantage point for a pacemaker and/or an active shunt for heart failure with preserved ejection fraction (“HFpEF”). In some embodiments, this can also be filled with a VSD septal occlusion nitinol device or foam device.

Any and/or all of the above-mentioned catheter pump devices/systems can be used in reverse in order to facilitate hemodynamic support to the RV and remove blood flow to titrate to help mapping and/or ablation. Any and/or all of these devices and methods can be used for all types of ablations such as ventricular tachycardia where hemodynamic support is necessary/needed. Any and/or all of these device and methods can also be used for complex coronary intervention procedures, structural heart disease cases such as percutaneous valve procedures, and the like. The above-mentioned processes and tools can be used in any combination, as well as in both directions to provide pump or suction support, as well as to increase or decrease blood flow to the heart to permit improve contact with tissue and ablation.

In some cases, irreversible electroporation can be delivered without the delivery of reversible electroporation. In some cases, the combination of electroporation and drug administration can reduce the concentration and/or amount of drug necessary to facilitate the same effects without electroporation. Such a decrease in amount and/or concentration of the drug administered can enhance the safety profile of the procedure. In some cases, the combination of electroporation and drug administration can reduce the energy required for electroporation. For example, drugs can be administered to specific tissues prior to electroporation. In some cases, the drug uptake can lower the threshold for cells to be ablated. Such a lowered threshold can enhance the safety and efficacy of ablation. In some cases, by lowering the threshold for electroporation, and/or ablation, the specific tissues can be more readily targeted, as the energy for electroporation would be sub-threshold when compared to surrounding tissue due to the differential of drug uptake.

In some cases, various methods can be used to increase time and/or surface area contact to allow for maximum cellular uptake of the drug. In some cases, such methods can alleviate or reduce the concern for drug washout through the systemic circulation from a beating heart. In some cases, a drug can be used to slow and/or stop the heart to allow for more time for drug activity. In some cases, the catheter assembly can include an adaptable catheter tip to enhance tissue-catheter contact. For example, the catheter tip can create a vacuum seal between the tissue and the catheter tip to allow for selective drug delivery and prevention of drug washout in the circulation. In some cases, the catheter tip can be deflectable to aid in positioning the catheter tip in proximity with the tissue. These can be used in conjunction and at the time of the percutaneous support to permit filling of the desired heart chamber with drug, expiration of specified amount of time, and then removal of the drug to reduce systemic toxicity.

In some cases, the systems and methods provided herein can provide reversible and transient termination of conduction in response to specific times when an arrhythmia takes place. Such a configuration can be an alternative form of defibrillation. For example, a drug delivery system can be attached to, or in communication with, the heart and can release a Purkinje specific drug and a low-level current (e.g., DC current) to permit Purkinje specific penetration of the therapeutic agent. In some cases, systemic effects can be reduced using a combination of electroporation and drug delivery. In some cases, the Purkinje specific drug can be reversible in the effect caused by the drug but allow for acute termination of the arrhythmia. In some cases, the system can include a mesh like portion with penetrating ports that are applied epicardially to the heart or a network applied along the endocardial surface with the capacity to deliver either a fluid or semi-fluid agent as well as a DC electric current.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described herein as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

1. A catheter device comprising:
a catheter shaft;
a balloon member attached at a distal end portion of the catheter shaft;
one or more electrodes on an outer surface of the balloon or in an interior of the balloon member and configured to deliver pulsed-electric field non-thermal electroporation ablation energy to treat ventricular fibrillation of a heart; and
a control system configured to adjust a pressure within the balloon member.

2. The catheter device of claim 1, wherein the control system is configured to pressurize and depressurize the balloon member in synchronization with an ECG of the heart or based on a rate of change of pressure in a left ventricular cavity of the heart.

3. (canceled)

4. (canceled)

5. The catheter device of claim 1, wherein the control system is configured to pressurize and depressurize the balloon member based on impedance changes in the heart or based on a timing cycle.

6. The catheter device of claim 1, wherein the control system is configured to pressurize and depressurize the balloon member based on impedance changes of a target tissue so as to automatically shut-off when ablation is completed.

7. The catheter device of claim 1, wherein the control system is configured to pressurize and depressurize the balloon member based on a timing in order to inflate for a period and deflate for a specified time in order to allow for adequate perfusion of the heart and maintenance of blood pressure.

8. The catheter device of claim 1, wherein the control system is configured to pressurize and depressurize the balloon member in coordination with the heart's systole/diastole in order to augment ventricular output.

9. The catheter device of claim 1, wherein the balloon member comprises an inner balloon with pores that is configured to inflate to a certain desired amount.

10. The catheter device of claim 1, wherein the balloon member comprises a porous material in order to not constrict or impede blood flow in the heart that would otherwise lead to hemodynamic instability.

11. A method for treating ventricular fibrillation of a heart, the method comprising:
using one or more of the catheter devices of claim 1 to deliver pulsed-electric field non-thermal electroporation ablation energy to one or more target locations of the heart.

12. The method of claim 11, wherein the one or more target locations of the heart includes a right stellate ganglion, a left stellate ganglion, or both.

13. The method of claim 11, wherein the one or more target locations of the heart includes an endocardial space of the heart, a mid-myocardium of the heart, and an epicardial space of the heart.

14. The method of claim 11, wherein the one or more target locations of the heart includes an arch of an aorta of the heart and associated nerve/ganglia tissue.

15. The method of claim 11, wherein the one or more target locations of the heart includes around great arteries of the heart and associated nerve ganglia tissue or an azygous vein connected to the heart.

16. The method of claim 11, wherein the one or more target locations of the heart includes that within a pericardial sac of the heart and in an oblique or transverse sinus of the heart or a structure within the superior mediastinum that contains the heart.

17. (canceled)

18. (canceled)

19. The method of claim 11, wherein the one or more target locations of the heart includes an aorta of the heart or a superior intercostal vein, and wherein the method also includes placing one or more electrodes in a left or right mainstem bronchus.

20. (canceled)

21. The method of claim 11, wherein the method also includes placing one or more electrodes in a trachea or endotracheal tube.

22. A method of pace termination for treating ventricular fibrillation (VF) of a heart, the method comprising delivering pacing to one or more target locations of the heart during one or more specific timings of the VF, wherein the pacing delivered is based on the target locations and a time of the VF and duration of VF.

23. The method of claim 22, wherein the one or more target locations of the heart includes an endocardium of the heart; mid-myocardium of the heart, and an epicardium of the heart.

24. The method of claim 22, wherein the one or more target locations of the heart includes an interventricular septum of the heart, and the pacing is delivered using a screw-in coil positioned longitudinally into the interventricular septum.

25. The method of claim 22, wherein the one or more target locations of the heart includes one or more of: (i) a distal coronary artery of the heart, (ii) a distal coronary venous branch, (ii) a mid-myocardium of the heart, (iv) a papillary muscle of the heart, (v) a moderator band of the heart, (vi) a false tendon of the heart.
